(19) United States
(12) Patent Application Publication
(10) Pub. No.: US 2026/0126452 A1
(43) Pub. Date: May 7, 2026

(54) SYSTEM AND METHOD FOR QUANTITATIVE PATHOLOGY USING MASS SPECTROMETRY IMAGING

(71) Applicants: The Brigham and Women’s Hospital, Inc., Boston, MA (US); President and Fellows of Harvard College, Cambridge, MA (US)

(72) Inventors: Nathalie Agar, Newton, MA (US); Sankha S. Basu, Boston, MA (US); Sylwia A. Stopka, Boston, MA (US); Sandro Santagata, Boston, MA (US); Marcia C. Haigis, Winchester, MA (US); Michael Regan, Boston, MA (US); Gerard Baquer, Boston, MA (US)

(21) Appl. No.: 18/705,692

(22) PCT Filed: Oct. 31, 2022
(86) PCT No.: PCT/US2022/048392

§ 371 Date: Apr. 29, 2024

Related U.S. Application Data
(60) Provisional application No. 63/273,863, filed on Oct. 29, 2021.

(51) Int. Cl. G01N 33/68; G01N 1/36; H01J 49/00

(52) U.S. Cl. CPC G01N 33/6851; G01N 1/36; H01J 49/0004; H01J 49/0009; G01N 2001/368; G01N 2458/15

(57) ABSTRACT

An apparatus for facilitating quantitative anatomic pathology using mass spectrometry imaging includes a solid support having at least one flat surface, a tissue homogenate having a thickness mounted to the at least one flat surface of the solid support, and a quantitative array having a thickness and comprising a tissue microarray having a plurality of wells and a series of varying concentrations of an isotopically labeled metabolite deposited in the plurality of wells. The quantitative array is mounted over the tissue homogenate on the solid support.

### CROSS-REFERENCE TO RELATED APPLICATIONS

This application is based on, claims priority to, and incorporates herein by reference in its entirety U.S. Ser. No. 63/273,863 filed Oct. 29, 2021, and entitled “MALDI MSI Quantitative Anatomic Pathology.”

### STATEMENT REGARDING FEDERALLY SPONSORED RESEARCH

This invention was made with government support under Grant Nos. CA201469, CA210180 and EB028741, awarded by National Institutes of Health. The government has certain rights in the invention

### FIELD

The present disclosure relates generally to quantitative mass spectrometry and, more particularly, to systems and methods for quantitative anatomic pathology using mass spectrometry imaging, for example, simultaneous quantitation of multiple biomarkers and drugs from tissue sections.

### BACKGROUND

Altered cellular metabolism is a hallmark of many different cancers and has proven to be a promising source of therapeutic targets. Exploiting these vulnerabilities, however, requires accurate metabolic characterization in heterogeneous specimens, which are often composed of neoplastic, stromal, and immune cells in a complex tumor microenvironment. Although liquid chromatography coupled to tandem mass spectrometry (LC-MS/MS) remains the gold standard for many analytical applications, most methods involve liquid matrices such as serum, plasma, or urine. Tissue-based quantification of small molecules using LC-MS/MS requires extensive sample preparation, making it challenging to implement in clinical laboratories. Furthermore, since these approaches involve homogenization or other tissue destructive processes, knowledge of spatial variability is lost, an important characteristic of endogenous metabolites in heterogeneous tumors.

Matrix-assisted laser desorption ionization mass spectrometry imaging (MALDI MSI) is a powerful analytical technique that provides spatially-preserved detection and quantification of analytes in tissue specimens. For example, MALDI-MSI can provide a powerful platform to map the spatial distribution of large biopolymers, proteins, peptides, lipids, small molecules, and drugs directly in tissue sections. Accurate and reliable quantification using MALDI-MSI, however, has faced several technical challenges. One challenge, common to most MS-based techniques, is variable ionization efficiency and ion suppression due to matrix effects. This has been addressed in LC-MS/MS methods by creating calibration curves spiked into comparable biological matrices as well as through normalization to spiked stable isotope internal standards (IS). However, matrix effect challenges are considerably more challenging when mapping metabolites in tissue specimens due to wide pixel-to-pixel variability in tissue composition and the lack of chromatographic separation.

Several approaches to account for matrix effects and to allow for better accuracy and precision have been applied to MALDI-MSI. For example, to generate calibration curves, the tissue mimetics may be used for the absolute quantitation for MALDI-MSI. The first mimetic approach consisted of spiking a range of different drug concentrations into a set of tissue homogenates that were pre-weighed into microcentrifuge tubes and then transferred into a home-built mold. This was further refined by creating cylindrical molds consisting of layers of serially frozen spiked-tissue homogenates in increasing concentrations to create calibration curves. Despite these improvements, both methods are still arguably cumbersome to generate, and require considerable space on a relatively small MALDI slide, leaving minimal room for experimental samples. In addition to the use of calibration curves, stable isotope internal standards have also been applied, though most often to analyze drugs in tissue, and less commonly for endogenous metabolites.

It would be desirable to provide a system and method for quantitative anatomic pathology using mass spectrometry imaging that can simultaneously quantitate a plurality of biomolecules and drugs with improved precision, accuracy and analytical characteristics.

### SUMMARY

In accordance with an embodiment, an apparatus for facilitating quantitative anatomic pathology using mass spectrometry imaging includes a solid support having at least one flat surface, a tissue homogenate having a thickness mounted to the at least one flat surface of the solid support, and a quantitative array having a thickness and comprising a tissue microarray having a plurality of wells and a series of varying concentrations of an isotopically labeled metabolite deposited in the plurality of wells. The quantitative array is mounted over the tissue homogenate on the solid support

In accordance with another embodiment, a method for making an apparatus for facilitating quantitative anatomic pathology using mass spectrometry imaging includes applying a material solution to a tissue microarray (TMA) to form an imprint mold having a plurality of wells, dispensing a series of varying concentrations of an isotopically labeled metabolite onto the plurality of wells to form a quantitative array, sectioning the quantitative array to produce a section with a predetermined thickness, sectioning a tissue homogenate to produce a section with a predetermined thickness, mounting the section of the tissue homogenate to a solid support having at least one flat surface, and mounting the section of the quantitative array over the tissue homogenate on the solid support.

In accordance with another embodiment, a method for quantitative anatomic pathology using mass spectrometry imaging includes preparing a test slide comprising a tissue homogenate, a quantitative array mounted over the tissue homogenate and comprising a plurality of wells and a series of varying concentrations of an isotopically labeled metabolite deposited in the plurality of wells, and a tissue sample mounted on the test slide adjacent to the quantitative array. The method further includes performing mass spectrometry imaging on the test slide to generate a set of mass spectrometry data and a set of calibration curves for the isotopically labeled metabolites, generating quantitative data for a plurality of metabolites based on the mass spectrometry data and the set of calibration curves; and generating a report comprising at least the quantitative data for the plurality of metabolites.

### BRIEF DESCRIPTION OF THE DRAWINGS

The present disclosure will hereafter be described with reference to the accompanying drawings, wherein like reference numerals denote like elements.

FIG. 1 is an illustration of an exploded view of an apparatus for quantitative anatomic pathology in accordance with an embodiment;

FIG. 2 illustrates a method for making a quantitative array in accordance with an embodiment;

FIG. 3 illustrates a method for making a tissue homogenate in accordance with an embodiment;

FIG. 4 illustrates a method for making a test slide for quantitative anatomic pathology in accordance with an embodiment;

FIG. 5 illustrates a process for quantitative anatomic pathology using mass spectrometry imaging in accordance with an embodiment; and

FIG. 6 illustrates a method for quantitative anatomic pathology using mass spectrometry imaging in accordance with an embodiment.

### DETAILED DESCRIPTION

The present disclosure describes a system and method for quantitative anatomic pathology using mass spectrometry imaging. In some embodiments, the mass spectrometry imaging is matrix assisted laser desorption ionization mass spectrometry imaging (MALDI MSI). In some embodiments, a quantitative array (or mimetic) formed from a tissue microarray (TMA) mold (e.g., an imprint mold) filled with serial dilutions of different isotopically-labeled analyte (e.g., metabolites, endogenous metabolites) standards may be cryosectioned onto a tissue homogenate on a slide and used to create calibration curves. The tissue array mold (e.g., an imprint mold) may be formed from gelatin, agar, or other materials with similar properties such as texture, freezing temperature and the ability to minimize diffusion from the serial dilutions. To improve precision and accuracy, in some embodiments, pixels outside of each quantitative array well may be automatically removed for analysis of the mass spectrometry data acquired from the slide. In some embodiments, the mass spectrometry imaging may be a MALDI MSI technique, and a second stable isotope internal standard (IS) may be incorporated in a matrix applied over the slide (including the quantitative array) and may be used for intensity normalization. In some embodiments, an internal standard (IS) may be applied directly to the tissue when no MALDI matrix is required (e.g., when the mass spectrometry technique uses ambient ionization). By using different stable isotope internal standards, one for normalization and another for calibration curves, the disclosed system and method can provide a more robust platform to quantify analytes, such as endogenous metabolites, that are naturally present in all or most tissues. In some embodiments, a normalization to matrix peaks may be used for analysis of the mass spectrometry data. Normalization to an IS or to matrix peaks can provide considerable improvement in precision and accuracy.

In some embodiments, the system and method may provide quantitative imaging of multiple biomarkers and drugs in tissue specimens for clinical and research applications. Advantageously, the system and method may simultaneously quantitate multiple biomolecules and drugs from tissue sections by MSI. Mass spectrometry imaging provides images from 100s-1000s of molecules from tissue sections and having the ability to simultaneously quantitate them can advantageously provide unprecedented molecular diagnostic information in a rapid manner (minutes).

The disclosed quantitative array can enable the quantification of several metabolites (e.g., endogenous metabolites) over a wide dynamic range, and can significantly improve over current approaches for quantitative mass spectrometry including, for example, MALDI MSI. The disclosed quantitative array can advantageously reduce the space needed on the mass spectrometry slides for calibration standards by approximately 90%. The disclosed quantitative array can improve the analytical characteristics and practical feasibility of mass spectrometry imaging (e.g., MALDI MSI) metabolite quantification in clinical and translational applications.

In some embodiments, mass spectrometry imaging (e.g., MALDI MSI) using the quantitative array on the test slide for calibration may be used to quantify a plurality of metabolites simultaneously and directly from biological tissues. In addition, the disclosed quantitative array may be used to quantify a plurality of purine metabolites in clinical tumor specimens using a single mass spectrometry imaging slide. In some embodiments, the quantitative array may be used to establish clinically quantitative imaging of related metabolites to aid in developing and assessing the effects of new therapeutics, for example, for glioblastoma. Accurate and precise mapping of endogenous metabolites in complex tumor specimens can be critical for clinical diagnostics and in understanding fundamental disease pathogenesis and tumor heterogeneity.

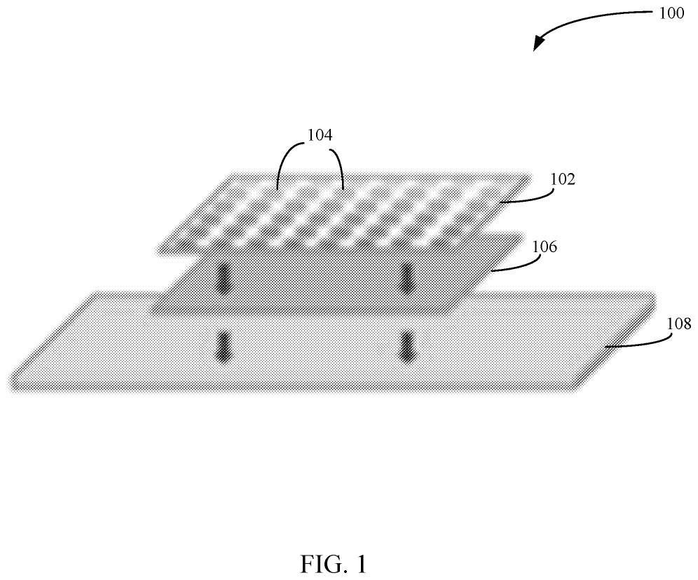

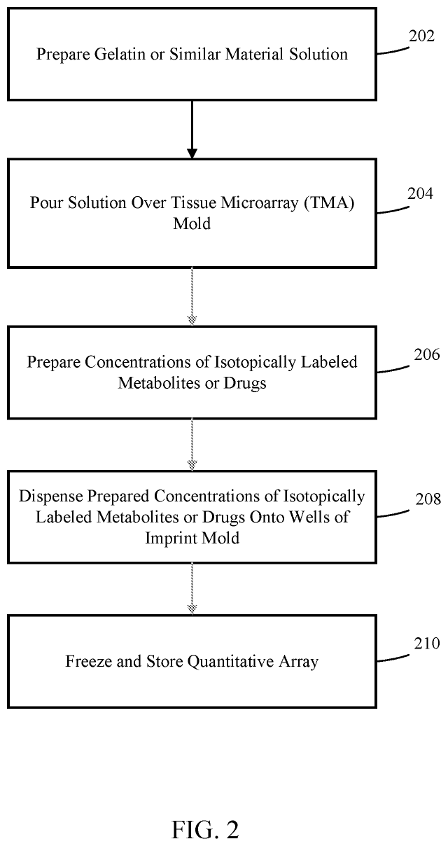

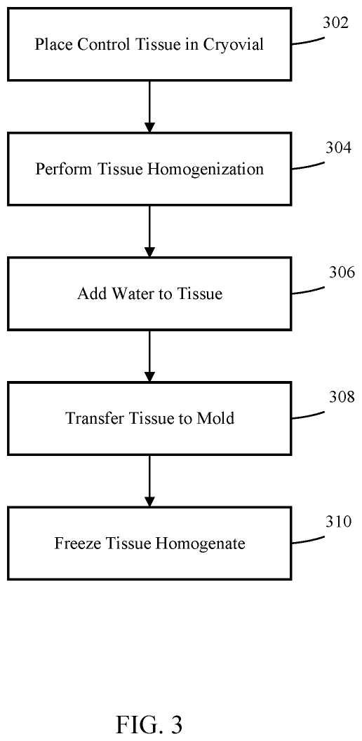

While the following description of FIGS. 1-6 is discussed in terms of MALDI mass spectrometry imaging, it should be understood that the system and method described herein, including the quantitative array, may be used for other types of mass spectrometry imaging including, for example, mass spectrometry techniques that utilize ambient ionization. FIG. 1 is an illustration of an exploded view of an apparatus for quantitative anatomic pathology in accordance with an embodiment. In FIG. 1, a test slide 100 for mass spectrometry imaging analysis may be prepared to include a quantitative array 102 (or mimetic) and a tissue homogenate 106. The quantitative array 102 and tissue homogenate 106 may be mounted on a solid support 108, for example, a slide, wafer, or other object on which samples such as tissues can be mounted for mass spectrometry analysis. In some embodiments, the quantitative array 104 is formed using a tissue microarray (TMA) mold and a material such as gelatin, agar, or other material with similar properties as gelatin or agar such as texture, freezing temperature, and the ability to minimize diffusion from serial dilutions. The quantitative array 102 can include a plurality of wells (or channels) 104 having a core and a depth. For example, in some embodiments, the quantitative array 102 may have 120 wells, each with a 1.5 mm core and a 10 μL volume. The wells or channels 104 of the quantitative array 102 may be filled with serial dilutions of isotopically labeled analyte standards (e.g., metabolite standards). The quantitative array 102 and the tissue homogenate 106 (e.g., a section of the quantitative array 102 and a section of the tissue homogenate 106) may be mounted (e.g., thaw mounted) to a flat surface of the slide 108. In some embodiments, the quantitative array 102 is advantageously mounted (e.g., thaw mounted) over the tissue homogenate 106. In some embodiments, the quantitative array 102 and the tissue homogenate 106 have the same thickness, for example, 10 μm. Example methods for preparing (or forming) the quantitative array 102 and the tissue homogenate 106 are described further below with respect to FIGS. 2 and 3, respectively.

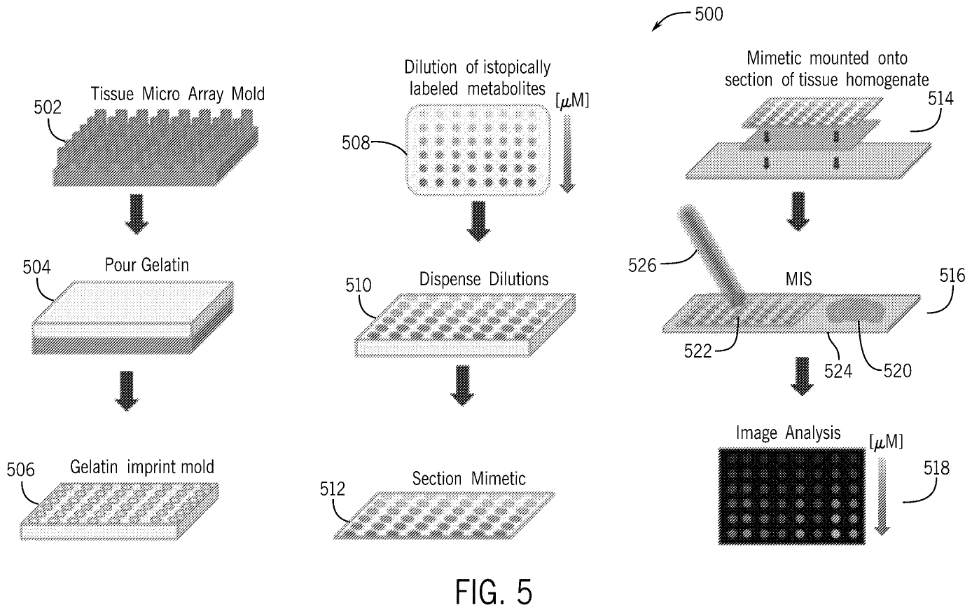

The quantitative array 102 may be used to provide a multi-standard model that can be prepared for panels of molecules for quantitation. For mass spectrometry imaging, a tissue sample (e.g., tissue sections) may be mounted to the slide 108 adjacent to the quantitative array 102 and tissue homogenate 104 as shown in FIG. 5 (step 516). In some embodiments, the slide may be a standard microscopy glass slide. In some embodiments, the test slide 100 with the quantitative array 102 and the tissue homogenate 106 may be used in a MALDI MSI process to create calibration curves for quantitative analysis of a plurality of metabolites. In some embodiments, the slide 108 may include a MALDI compatible surface, for example, the slide 108 may be an Indium tin oxide (ITO)-coated slide. For MALDI MSI, a matrix may also be applied to the slide 108, for example, over both the quantitative array 102 and the tissue sample (not shown).

In some embodiments, the quantitative array 102 may be configured to minimize the area on a slide 108 required for calibration or quality control (QC) and to maximize the space available on the slide 108 available for the tissue specimens (e.g., tissue sample 520 shown in FIG. 5) to be analyzed. Some previous methods for quantitative mass spectrometry imaging utilized seven wells per analyte and employed approximately 20% of a slide which hindered QC as three times replicate mimetics would cover more than half of the slide and leave little space for tissue samples (e.g., tissue sections). By using TMA molds, quantitative array 102 advantageously can fit more wells per analyte in less space, for example, nine wells per analyte in only approximately 2% of a slide. In this example, three replicate mimetics for QC measurements take up approximately 6% of the slide, leaving ample space for tissue sections on the slide. In some embodiments, the space reduction (e.g., the 10-fold space reduction in the described example) allows the quantitative array 102 to multiplex analytes (e.g., metabolites). For example, a quantitative array 102 containing 150 wells (15 rows×10 columns) can quantify 15 analytes with ten varied concentrations in a single mass spectrometry imaging acquisition. Accordingly, this can advantageously enable quantification using mass spectrometry imaging to cover multiple intermediate metabolites within a pathway. Additionally, the reduction in space provided by the disclosed quantitative array 102 ensures that all quantification and QC can be run along with the tissue samples, which can result in blocking batch effects and ensuring accurate and precise quantification.

FIG. 2 illustrates a method for making a quantitative array in accordance with an embodiment. At block 202, a material solution formed using a material such as, for example, a gelatin, agar, or other material with similar properties may be prepared. In some embodiments, the material solution may be a 40% gelatin solution and may be autoclaved. A 40% gelatin solution may be cryosection more uniformly and may prevent the creation of breaks in the mold upon sectioning. At block 204, the material solution may be poured onto a tissue microarray (TMA) mold to create an imprint mold (e.g., a gelatin imprint mold). The tissue microarray mold can include a plurality of wells (or channels) and each well may have a core and a volume. For example, in some embodiments, the tissue microarray mold may have 120 wells, each with a 1.5 mm core and a 10 μL volume. In some embodiments, the material used for the tissue microarray imprint mold may be selected to minimize the diffusion from the serial dilutions that are dispensed into the tissue microarray imprint mold (e.g., an imprint mold formed from the selected material such as gelatin, agar, etc.).

At block 206, varied concentrations of isotopically labeled metabolites or drugs may be prepared for creating a multi-standard quantitative array. The standards may be isotopically labelled to distinguish from biomolecules in the homogenate to which they are applied. The analyte (e.g., metabolite) standards may be diluted to different concentrations in a dilution solvent, for example, water, agarose, gelatin, or collagen. For example, a 50 mM concentration stock may be prepared from isotopically ¹⁵N labeled glutamate in water. A dilution series may be made in collagen to obtain final concentrations ranging from 0.05 mM to 20 mM. Similarly, isotopically labeled adenosine-¹⁵N₅ 5′-triphosphate (ATP) disodium salt solution, adenosine-¹⁵N₅ 5′-diphosphate disodium salt (ADP), adenosine-¹³C₁₀¹⁵N₅ 5′-monophosphate (AMP) disodium salt, and adenosine may be diluted from a 100 mM stock solution to obtain final concentrations ranging from 0.005-50 mM solutions. In some embodiments, collagen may be used as the solvent to reduce analyte (e.g., metabolite) diffusion between the wells of the gelatin imprint mold.

At block 208, the prepared concentrations of the isotopically labeled metabolites or drugs may be dispensed onto the wells (or channels) of the TMA imprint mold with the selected material such as, for example, gelatin (i.e., the gelatin imprint mold). A predetermined amount of each concentration may be dispensed onto a designated well (or channel) of the imprint mold (e.g., a gelatin imprint mold). For example, 10 μL of each concentration may be dispensed onto the imprint mold wells at −20° C. in a cryostat. In another example, the final metabolite concentrations may be pipetted onto the wells in a cryostat at a temperature of −15° C. to −20° C. to, for example, prevent the metabolite concentrations from freezing during deposition. At block 210, the quantitative array (or mimetic) may be stored in a freezer (e.g., −80° C.) until the preparation of a test slide for mass spectrometry (e.g., MALDI MAI) analysis. As discussed above, the quantitative array may be applied to a tissue homogenate adjacent to tissue sections of interest for quantitative imaging.

The quantitative array (or mimetic) is versatile and can be used to quantitate different biological tissue. Advantageously, the quantitative array (or mimetic) can be multiplexed. For example, an array (or mold) that has 15 rows and 10 columns can contain 10-15 different metabolites on one mass spectrometry imaging (e.g., MALDI-MSI) run. This can allow quantitation of different metabolic pathways as the intermediate metabolites can be in different rows. For example, for purine metabolism pathway, using this quantitative array (or mimetic), can cover about 70% of the metabolites of this pathway. In some embodiments, the quantitative array may be configured to use three to six replicates.

As discussed above, a quantitative array 102 (shown in FIG. 2) may be configured to minimize the area on a slide (e.g., slide 108) required for calibration or quality control (QC) and to maximize the space available on the test slide available for the tissue specimens (e.g., tissue sample 520 shown in FIG. 5) to be analyzed. By using TMA molds, quantitative array 102 advantageously can fit more wells per analyte in less space, for example, nine wells per analyte in only approximately 2% of a slide. In this example, three replicate mimetics for QC measurements take up approximately 6% of the slide, leaving ample space for tissue sections on the slide. In some embodiments, the space reduction (e.g., the 10-fold space reduction in the described example) allows the quantitative array to multiplex analytes. For example, a mold containing 150 wells (15 rows×10 columns) can quantify 15 analytes with ten varied concentrations in a single mass spectrometry imaging (e.g., MALDI-MSI) acquisition. Accordingly, this can advantageously allow quantification using mass spectrometry imaging (e.g., MALDI-MSI) to cover multiple intermediate metabolites within a pathway. Additionally, the reduction in space provided by the disclosed quantitative array 102 ensures that all quantification and QC can be run along with the tissue samples, which can result in blocking batch effects and ensuring accurate and precise quantification.

FIG. 3 illustrates a method for making a tissue homogenate in accordance with an embodiment. At block 302, a predetermined amount (e.g., 10 g) of a control tissue (e.g., human brain tissue) may be placed in a cryovial (e.g. a 15 mL cryovial) and at block 304, tissue homogenization may be performed at, for example, room temperature using a homogenizer for a predetermined amount of time, for example, 60 seconds. Once the tissue is completely homogenized, at block 306 water (e.g., 20%) may be added such that the tissue becomes fluent enough to transfer using a syringe. In some embodiments, the addition of water can minimize the holes in the tissue that can be generated while sectioning. In some embodiments, the tissue homogenate may be blended for an additional amount of time, for example, another 60 seconds. At block 308, the tissue may then be transferred to a mold (e.g., a plastic rectangular mold) and at block 308 frozen and then stored at −80° C. until the preparation of a test slide for mass spectrometry (e.g., MALDI MSI) analysis. As discussed above, a quantitative array may be applied to a tissue homogenate adjacent to tissue sections of interest for quantitative imaging.

Advantageously, in some embodiments, the tissue homogenate is not spiked with analyte (e.g., metabolites). Previous quantitative mass spectrometry methods have relied on analyte-spiked tissue homogenates which locked each preparation to a particular species or tissue type. By decoupling the analyte (e.g., metabolite) dilutions from the tissue homogenate, the disclosed quantitative array can be generalized to any species or tissue type. The same quantitative array can be sectioned on demand and placed over the required tissue homogenate. This can facilitate bulk analyses by reducing preparation time and technical variability, as well as increasing stability.

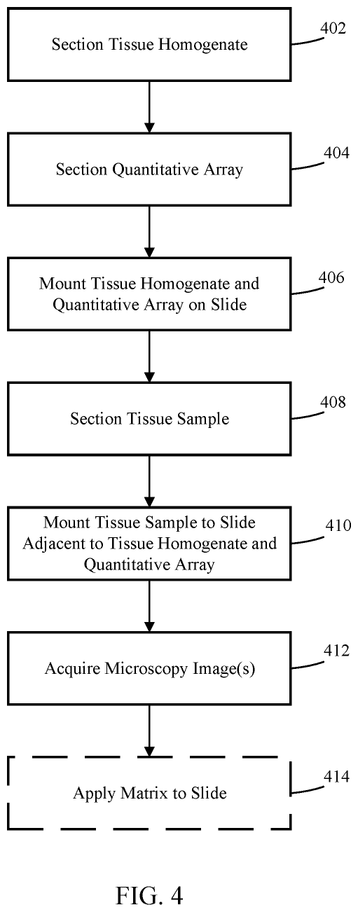

As discussed above with respect to FIG. 1, the test slide 100 with a quantitative array 102 mounted over a tissue homogenate 104 on a slide 108 may be used in a mass spectrometry imaging (e.g., MALDI MSI) process to create calibration curves for quantitative analysis of a plurality of metabolites. For mass spectrometry imaging, the test slide may be further prepared for mass spectrometry analysis by mounting a tissue sample (e.g., tissue sections) to the slide 108 adjacent to the quantitative array 102 and tissue homogenate 104. For MALDI MSI, the slide 108 may be further prepared by applying a matrix as shown in FIG. 5 (step 516). FIG. 4 illustrates a method for making a test slide for quantitative anatomic pathology in accordance with an embodiment. At block 402, a tissue homogenate (e.g., a frozen tissue homogenate) may be sectioned and at block 404, a quantitative array (e.g., a frozen quantitative array) may be sectioned. In some embodiments, the quantitative array and the tissue homogenate may be sectioned at the same thickness (e.g., 10 μm) as the tissue sample to be analyzed. As mentioned above, the tissue homogenate may be selected based on the tissue of interest for analysis. For example, the tissue homogenate may be the same species and organ as the tissue of interest. At block 406, the quantitative array (or mimetic) may be mounted over the tissue homogenate on the same slide. For example, the tissue homogenate may be sectioned and thaw mounted to the slide and then the quantitative array may be sectioned and thaw mounted over the tissue homogenate. In some embodiments, the test slide may be an Indium tin oxide (ITO)-coated slide or a standard microscopy glass slide.

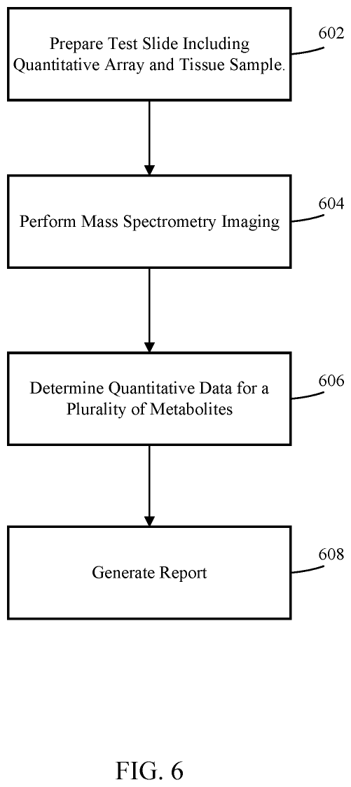

At block 408, one or more tissue samples (e.g., a frozen tissue sample) of the tissue of interest for analysis and quantitation may be sectioned. The tissue sample is sectioned at a thickness (e.g., 10 μm) and at block 410 may be mounted to the same slide as the quantitative array and tissue homogenate. Step 516 of the process 500 of FIG. 5, discussed further below, illustrates a tissue sample 520 mounted adjacent to a quantitative array 522 on a slide 524. In some embodiments, the tissue samples may be thaw mounted to the slide. As mentioned, in some embodiments, the test slide may be an indium tin oxide (ITO)-coated slide or a standard microscopy glass slide. In some embodiments, the tissue sample(s) may be placed in a cryostat at −20° C. (e.g., for 15 minutes) to allow thermal equilibration. In some embodiments, a serial section may be obtained for staining (e.g., hematoxylin and eosin (H&E) staining). On the quantitative array, tissue homogenate and tissue sample(s) are mounted to the slide. In some embodiments, the slide may be allowed to dry under a vacuum desiccator. At block 412, microscopy images of the slide may be acquired. For example, optical microscopy images of a slide may be acquired using a bright field microscope using a 20× magnification. In some embodiments, other techniques may be used to acquire microscopy images including, for example, fluorescence microscopy, bright field light microscopy, reflectance microscopy, combinations therefore, and the like. In some embodiments, an internal standard (IS) may be applied to the tissue when no MALDI matrix is required (e.g., when the mass spectrometry technique utilizes ambient ionization). The internal standard applied to the tissue may be used, for example, for normalization as described further below with respect to FIG. 6.

At block 414, if the mass spectrometry method being utilized is MALDI MSI a matrix may be applied or deposited on the entire slide (i.e., both the quantitative array and the tissue sample. The matrix can be a chemical compound capable of crystallizing and that transfer energy from incident laser light to sample molecules. In some embodiments any MALDI matrix material known in the art may be used. For example, a 1,5 diaminonaphthalene (DAN)-HCl matrix solution may be prepared and used for metabolite quantitation. In an example, the 1, 5 DAN matrix solution may be prepared by dissolving 1,5-DAN (4.3 mg/mL) in 4.5/5.0/0.5 HPLC grade water/ethanol/1M HCl. In another example, a 2,5-dihydroxybenzoic acid (DHB) matrix solution may be prepared and used for drug quantitation. In an example, the 2,5 DHB matrix solution may be prepared by dissolving DHB (160 mg/mL) in 70:30 methanol:0.1% TFA with 1% DMSO. The matrix may be deposited using known methods. In some embodiments, the matrix may deposited using a spraying technique. Other matrix deposition techniques can include, for example, solvent evaporation deposition, spin coating, blade deposition, and chemical printing. In some embodiments, the matrix solution may also include an internal standard material (e.g., D-Glutamate-d5) that may be used for normalization as discussed further below with respect to FIG. 6.

FIG. 5 illustrates a process for quantitative anatomic pathology using mass spectrometry imaging in accordance with an embodiment. The process 500 illustrates an example workflow for metabolite and drug quantification using a quantitative array (or mimetic) for generating calibration curves for mass spectrometry imaging analysis. At steps 502-506, an imprint mold (e.g., a gelatin imprint mold) is created from a tissue microarray (TMA) mold by pouring a material solution (e.g., a solution formed using a material such as gelatin, agar or other material with similar properties) onto the tissue microarray mold. The tissue microarray mold can include a plurality of wells (or channels) and each well may have a core and a volume. For example, in some embodiments, the tissue microarray mold may have 120 wells, each with a 1.5 mm core and a 10 μL volume. At step 508, varied (e.g., serial) concentrations of isotopically labeled metabolites or drugs may be prepared and then at step 510 are dispensed into the channels or wells of the imprint mold to create a quantitative array (or mimetic). In some embodiments, the concentrations of isotopically labeled metabolite may be dispensed onto the wells of the quantitative array in a cryostat (e.g., at −20° C.). The quantitative array (or mimetic) may then be frozen (e.g., at −20° C.) and at step 512 sectioned (e.g., at 10 μm thickness). At step 514, the quantitative array may be mounted over a human tissue homogenate that is sectioned at the same thickness. The quantitative array 522 (and tissue homogenate) may be mounted to a slide 524 adjacent to a tissue sample 520 as shown at step 516 and at step 518 the slide 524 (with quantitative array 522 and tissue sample 520) may be analyzed using mass spectrometry imaging. In some embodiments, the mass spectrometry imaging technique may be MALDI MSI. For MALDI MSI, the whole slide 524 (quantitative array 522 and tissue sample 520) may then be sprayed with the desired matrix and at step 518 analyzed using MALDI-MSI. For the matrix-assisted laser desorption ionization, the test slide 524 with the applied photon-absorbing matrix may be exposed to a pulse of laser light 526 and the matrix transfers energy to the molecules in the standards and samples thereby promoting their desorption and ionization.

FIG. 6 illustrates a method for quantitative anatomic pathology in accordance with an embodiment. At block 602, a test slide for mass spectrometry imaging (e.g., MALDI MSI) analysis may be prepared with a quantitative array mounted on a slide adjacent to the tissue sample as described above with respect to FIGS. 1, 4 and 5. The prepared test slide may then be placed in or introduced to a mass spectrometry device and used for mass spectrometry imaging (e.g., MALDI MSI) analysis. At block 604, a mass spectrometry technique may be performed on the prepared test slide to acquire data (e.g., signals) which may then be analyzed. In some embodiments, the mass spectrometry imaging technique used to analyze the tissue sample and generate quantitative data may be a known MALDI MSI technique. In some embodiments, a Fourier Transform Ion Cyclotron Resonance (FT-ICR) mass spectrometry imaging technique may be performed using, for example, a 15 Tesla mass spectrometer with a dual ESI/MALDI source operating in negative ion mode. Other types of MSI include, for example, time of flight (TOF) MS, linear trap quadruple MS, and orbitrap MS. For the matrix-assisted laser desorption ionization, the test slide with the applied photon-absorbing matrix may be exposed to a pulse of laser light (e.g., laser 526 shown in step 516 of FIG. 5) and the matrix transfers energy to the molecules in the standards and samples thereby promoting their desorption and ionization.

Calibration curves are typically obtained by averaging all the acquired pixels within the selected region of interest (ROI) for each quantitative array. However, this selectin of an ROI by a user may induce two main sources of variability. Firstly, the ROI can include pixels that fall out of the quantitative array and thus do not contain calibrant. Secondly, the tissue homogenate typically presents cracks and imperfections that lead to inconsistencies in ionization. These two phenomena lead to overall inconsistencies in the pixel-wise intensities with each ROI which in turn affect the overall performance of quantification. In some embodiments, to address these challenges the mass spectrometry data may be processed to exclude pixels outside of the quantitative array wells or in tissue homogenate cracks. This can effectively improve both accuracy and precision.

In some embodiments, the mass spectrometry data may also be processed to perform outlier removal. For example, points with regression residuals greater than the (regression standard error)*(t-value at 95% confidence interval) may be considered outliers and thus removed.

In some embodiments, a known normalization method may be used for the mass spectrometry data such as, for example, no normalization, Total Ion Current (TIC), matrix peak and stable-isotope-labeled glutamate Internal Standard (IS). In some embodiments, a stable isotope labeled glutamate IS normalization method may be used and the IS included in the matrix applied to the test slide or, for MSI techniques that utilize ambient ionization, the IS may be applied directly to the tissue (e.g., using a spraying technique). In some embodiments, the mass spectrometry imaging technique may be MALDI MSI and the IS may be homogenously sprayed with the MALDI matrix over the tissue sections. In an example, a deuterium-labeled glutamate standard may be spiked into a DAN-HCl matrix and sprayed over the quantitative array and different normalization parameters may be applied. Advantageously, the addition of an isotopically labeled IS can provide improved precision, and accuracy. In some embodiments, a normalization to the matrix peak technique may be used. Utilizing an IS or normalizing to the matrix peak technique can correct for several factors and improve the precision of analysis which can be important when translating to a clinical setting.

Known calibration models may be used to fit to the replicates of each concentrate including, for example, linear regression (y=ax+b), weighted linear regression

$$( w = \frac{1}{{y}^{2}} )$$

and power regression (y=ax^{b}). In some embodiments, the power model may be used for concentration dilutions spanning multiple decades while the weighted linear may be used when only working with 1 decade.

At block 606, quantitative data for a plurality of metabolites may be determined based on the mass spectrometry (e.g., MALDI MSI) analysis (block 604). For example, quantitative data for a targeted analyte (e.g., a metabolite) may be determined by referencing the calibration curves obtained from signals generated by the quantitative array. At block 608, a report may be generated including, for example, the determined quantitative metabolite data.

The following example sets forth, in detail, ways in which the present disclosure was evaluated and ways in which the present disclosure may be used or implemented and will enable one of ordinary skill in the art to more readily understand the principles thereof. The following example is presented by way of illustration and are not meant to be limiting in any way.

Glioblastoma (GBM) is an aggressive primary brain tumor with a median survival of less than 15 months. The tumor microenvironment (TME) plays a critical role in tumor development, therefore, understanding the immunosuppressive TME could lead to improved immunotherapies. Research has shown that CD73, an ectoenzyme that functions in extracellular purinergic metabolism that generates adenosine, is highly expressed in various cancers and is a key regulator of the tumor-immune interactions. In this example, the disclosed quantitative array and workflow was applied to 14 clinical samples consisting of 12 meningiomas, one metastatic melanoma, and one solitary fibrous tumor. Serial tissue sections were stained with hematoxylin and eosin and analyzed using MALDI MSI. In this example, the quantitative array and workflow provided simultaneous metabolite quantification of biological tissue with up to 15 possible metabolites. For this example, the slide setup consisted of four purine metabolites: ATP, ADP, AMP, and adenosine in two rows for reproducibility next to the 14 clinical tissues. Importantly, the quantitative array (or mimetic) is placed on the same glass slide as the tissue section of interest to eliminate the risk of instrumental changes between cycles, and irregularities from sample/matrix preparation. After normalizing all data to the matrix peak, four plots were generated to aid in the quantitation. In this example, all metabolites showed good linearity of calibration: adenosine (R²=0.9908), ATP (R²=0.9876), ADP (R²=0.9229), AMP (R²=0.9997). The quantitation demonstrated very similar LOD values: adenosine (0.06 mM), ATP (0.01 mM), AMP (0.07 mM)), and ADP (0.17 mM). The same trend was found for LOQ values, (adenosine (0.19 mM), ATP (0.03 nM), AMP (0.23 mM)), with ADP at 0.56 nM. Using these values, the absolute metabolite concentration may be quantified with each tumor tissue a sample. The concentration of adenosine ranged from 0.06-1.67 mM, AMP ranged from 0.06 to 0.67 mM, ADP ranged from 0.15 to 1.02 mM, ATP ranged from 0.02 to 0.44 mM.

The clinical MSI tissue quantitation can be further applied to find correlations between established biomarkers such as CD73 and the respective metabolites. In this example, it was shown that high CD73 expression levels led to higher extracellular levels of adenosine. Furthermore, this MSI approach can be used to monitor oncometabolite such as 2-hydroxyglutarate (2-HG) concentrations in glioblastomas (GBM) expressing isocitrate dehydrogenase 1 (IDH1) variant. Correlations between biomarkers and respective metabolites can provide additional information on the TME to aid in the diagnosis of certain cancers and the development of new immunotherapies.

Computer-executable instructions for quantitative anatomic pathology using mass spectrometry imaging according to the above-described methods may be stored on a form of computer readable media. Computer readable media includes volatile and nonvolatile, removable, and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital volatile disks (DVD) or other optical storage, magnetic cassettes, magnetic tape, magnetic disk storage or other magnetic storage devices, or any other medium which can be used to store the desired instructions and which may be accessed by a system (e.g., a computer), including by internet or other computer network form of access

The present invention has been described in terms of one or more preferred embodiments, and it should be appreciated that many equivalents, alternatives, variations, and modifications, aside from those expressly stated, are possible and within the scope of the invention.

1. An apparatus for facilitating quantitative anatomic pathology using mass spectrometry imaging, the apparatus comprising:
a solid support having at least one flat surface;
a tissue homogenate having a thickness mounted to the at least one flat surface of the solid support; and
a quantitative array having a thickness and comprising a tissue microarray having a plurality of wells and a series of varying concentrations of an isotopically labeled metabolite deposited in the plurality of wells, wherein the quantitative array is mounted over the tissue homogenate on the solid support.

2. The apparatus according to claim 1, wherein the solid support is a slide.

3. The apparatus according to claim 1, wherein the tissue homogenate thickness is the same as the quantitative array thickness.

4. The apparatus according to claim 1, further comprising:
a tissue sample having a thickness and mounted to the at least one flat surface of the solid support and adjacent to the quantitative array.

5. The apparatus according to claim 4, further comprising:
a matrix assisted laser desorption ionization matrix deposited on the quantitative array and the tissue sample.

6. The apparatus according to claim 4, wherein the tissue sample thickness is the same as the quantitative array thickness.

7. A method for making an apparatus for facilitating quantitative anatomic pathology using mass spectrometry imaging, the method comprising:
applying a material solution to a tissue microarray (TMA) to form an imprint mold having a plurality of wells;
dispensing a series of varying concentrations of an isotopically labeled metabolite onto the plurality of wells to form a quantitative array;
sectioning the quantitative array to produce a section with a predetermined thickness;
sectioning a tissue homogenate to produce a section with a predetermined thickness;
mounting the section of the tissue homogenate to a solid support having at least one flat surface; and
mounting the section of the quantitative array over the tissue homogenate on the solid support.

8. The method according to claim 7, wherein the material solution is formed with a material configured to minimize diffusion of the series of isotopically labeled metabolites.

9. The method according to claim 7, wherein the predetermined thickness of the tissue homogenate is the same as the predetermined thickness of the quantitative array.

10. The method according to claim 7, further comprising:
sectioning a tissue sample to produce a section with a predetermined thickness; and
mounting the section of the tissue sample to the solid support adjacent to the quantitative array.

11. The method according to claim 10, further comprising:
applying a matrix assisted laser desorption ionization matrix on the section of the quantitative array and the section of the tissue sample.

12. The method according to claim 10, wherein the predetermine thickness of the tissue sample is the same as the predetermined thickness of the quantitative array.

13. The method according to claim 11, wherein the matrix includes an isotopically labeled internal standard.

14. A method for quantitative anatomic pathology using mass spectrometry imaging, the method comprising:
preparing a test slide comprising a tissue homogenate, a quantitative array mounted over the tissue homogenate and comprising a plurality of wells and a series of varying concentrations of an isotopically labeled metabolite deposited in the plurality of wells, and a tissue sample mounted on the test slide adjacent to the quantitative array;
performing mass spectrometry imaging on the test slide to generate a set of mass spectrometry data and a set of calibration curves for the isotopically labeled metabolites;
generating quantitative data for a plurality of metabolites based on the mass spectrometry data and the set of calibration curves;
generating a report comprising at least the quantitative data for the plurality of metabolites.

15. The method according to claim 14, wherein the mass spectrometry imaging is matrix assisted laser desorption ionization mass spectrometry imaging and the test slide further comprises a matrix assisted laser desorption ionization matrix applied to the quantitative array and the tissue sample

16. The method according to claim 15, wherein the matrix assisted laser desorption ionization mass spectrometry imaging comprises performing a Fourier Transform Ion Cyclotron Resonance (FT-ICR) mass spectrometry imaging technique.

17. The method according to claim 14, further comprising excluding mass spectrometry data located outside of the quantitative array wells.

18. The method according to claim 14, wherein the matrix includes an isotopically labeled internal standard for normalization.

19. The method according to claim 14, wherein the set of calibration curves are generated using a weighted linear regression model.

20. The method according to claim 14, further comprising performing normalization to the matrix peak.
